SEQUENCE LISTING (27 sequences)

SEQ ID NO: 1 - protein, 7 aa, synthetic construct
FPGSDRF

SEQ ID NO: 2 - protein, 9 aa, synthetic construct
RRRRRRRRR

SEQ ID NO: 3 - protein, 10 aa, synthetic construct
RRRRRRRRRF

SEQ ID NO: 4 - protein, 5 aa, synthetic construct
  REPEAT 2
AGRGD

SEQ ID NO: 5 - protein, 11 aa, synthetic construct
FPGSDRFGRGD

SEQ ID NO: 6 - protein, 14 aa, synthetic construct
FPGSDRFGGGGRGD

SEQ ID NO: 7 - protein, 20 aa, synthetic construct
FPGSDRFGGGGGGGGGGRGD

SEQ ID NO: 8 - protein, 11 aa, synthetic construct
FPGSDRFARGD

SEQ ID NO: 9 - protein, 12 aa, synthetic construct
FPGSDRFAGRGD

SEQ ID NO: 10 - protein, 15 aa, synthetic construct
FPGSDRFAGGGGRGD

SEQ ID NO: 11 - protein, 21 aa, synthetic construct
FPGSDRFAGGGGGGGGGGRGD

SEQ ID NO: 12 - protein, 11 aa, synthetic construct
FFPGSDRFRGD

SEQ ID NO: 13 - protein, 12 aa, synthetic construct
FFPGSDRFGRGD

SEQ ID NO: 14 - protein, 15 aa, synthetic construct
FFPGSDRFGGGGRGD

SEQ ID NO: 15 - protein, 21 aa, synthetic construct
FFPGSDRFGGGGGGGGGGRGD

SEQ ID NO: 16 - protein, 12 aa, synthetic construct
FFPGSDRFARGD

SEQ ID NO: 17 - protein, 13 aa, synthetic construct
FFPGSDRFAGRGD

SEQ ID NO: 18 - protein, 22 aa, synthetic construct
FFPGSDRFAGGGGGGGGGGRGD

SEQ ID NO: 19 - protein, 19 aa, synthetic construct
RRRRRRRRRFPGSDRFRGD

SEQ ID NO: 20 - protein, 29 aa, synthetic construct
RRRRRRRRRFPGSDRFGGGGGGGGGGRGD

SEQ ID NO: 21 - protein, 20 aa, synthetic construct
RRRRRRRRRFPGSDRFARGD

SEQ ID NO: 22 - protein, 30 aa, synthetic construct
RRRRRRRRRFPGSDRFAGGGGGGGGGGRGD

SEQ ID NO: 23 - protein, 20 aa, synthetic construct
RRRRRRRRRFFPGSDRFRGD

SEQ ID NO: 24 - protein, 30 aa, synthetic construct
RRRRRRRRRFFPGSDRFGGGGGGGGGGRGD

SEQ ID NO: 25 - protein, 21 aa, synthetic construct
RRRRRRRRRFFPGSDRFARGD

SEQ ID NO: 26 - protein, 31 aa, synthetic construct
RRRRRRRRRFFPGSDRFAGGGGGGGGGGRGD

SEQ ID NO: 27 - protein, 10 aa, synthetic construct
FPGSDRFRGD

(19) United States
(12) Patent Application Publication
(10) Pub. No.: US 2025/0152659 A1
(43) Pub. Date: May 15, 2025

(54) METHOD FOR PREVENTING OR TREATING PARKINSON'S DISEASE

(71) Applicant: Suzhou Mingren Pharmaceutical Biotechnology Co., Ltd., Suzhou (CN)

(72) Inventor: Jianwei ZHOU, Suzhou (CN)

(21) Appl. No.: 19/026,254

(22) Filed: Jan. 16, 2025

Related U.S. Application Data
(63) Continuation-in-part of application No. PCT/CN2022/138737, filed on Dec. 13, 2022.

(30) Foreign Application Priority Data
Sep. 16, 2022 (CN) 202211129238.5

(51) Int. Cl. A61K 38/08; A61P 25/16

(52) U.S. Cl. CPC A61K 38/08; A61P 25/16; C07K 2319/01

(57) ABSTRACT

A method for preventing or treating Parkinson's disease including administering a patient in need thereof a pharmaceutical composition including a polypeptide, the peptide having an amino acid sequence I or II: I: FPGSDRF (SEQ ID NO: 1)-Z; II: X-FPGSDRF (SEQ ID NO: 1)-Z; S represents phosphorylated serine; X and Z independently represents an amino acid or an amino acid sequence; X is selected from F, (R)₉ (SEQ ID NO: 2), (R)₉-F (SEQ ID NO: 3), 6-aminohexanoic acid, 6-aminohexanoic acid-F, 6-aminohexanoic acid-(R)₉ (SEQ ID NO: 2), 6-aminohexanoic acid-(R)₉-F (SEQ ID NO: 3); and Z is selected from (G)ₙ-RGD or A-(G)ₙ-RGD (SEQ ID NO: 4), where n is an integer greater than or equal to 0, in the range of 0-10.

### CROSS-REFERENCE TO RELATED APPLICATIONS

This application is a continuation-in-part of International Patent Application No. PCT/CN2022/138737 with an international filing date of Dec. 13, 2022, designating the United States, now pending, further claims foreign priority benefits to Chinese Patent Application No. 202211129238.5 filed Sep. 16, 2022. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, MA 02142.

### INCORPORATION-BY-REFERENCE OF SEQUENCE LISTING

This application contains a sequence listing, which has been submitted electronically in XML file and is incorporated herein by reference in its entirety. The XML file, created on Dec. 18, 2024, is named NJYK-00501-UUS.xml, and is 24,784 bytes in size.

### BACKGROUND

The disclosure relates to a method for preventing or treating Parkinson's disease.

Parkinson's disease (PD) is a common neurodegenerative disorder characterized by degeneration and loss of dopamine (DA) neurons in the substantia nigra compacta (SNc), reduction of DA transmitters in the striatum, and glial cell hyperplasia accompanied by the formation of intracellular eosinophilic Lewy body.

The pathogenesis of PD is still unclear, so PD remains an incurable disease. Clinical treatment mainly uses Levodopa (L-Dopa) to make up for the lack of dopamine (DA), thus alleviating the motor symptoms of PD. However, the therapy cannot cure PD or slow down the further development of the disease, and long-term use of the therapy will also cause a series of complications, such as L-Dopa-induced dyskinesias (LIDs), symptom fluctuations, and so on.

To study the mechanisms of DA neuronal injury leading to neurodegenerative lesions, the attention has been focused on damaged neurons. However, certain studies have shown that neighboring glial cells also play an important role in the death process of the neurons. Astrocytes are the most abundant glial cells in the mammalian brain and are an important component of the nervous system. Increasingly, abnormalities in astrocyte structure and function have been shown to play an important role in a variety of neurodegenerative diseases, brain injury, and central nervous system inflammation. Astrocytes maintain the extracellular environment, stabilize intercellular communication and mediate neuronal physiology and pathology. Astrocytes exert their functions largely dependent on the fact that they can release and take up molecules from the extracellular microenvironment, thus playing a protective or damaging role for neurons.

Compared to neurons, glial cells have a more robust antioxidant enzyme synthesis system and much higher levels of intracellular antioxidant enzymes and type II detoxification enzymes. One reason for the difference is that nuclear factor E2-related factor 2 (Nrf2) is preferentially activated in astrocytes compared to neurons. The activation of Nrf2 allows it to bind to genes with antioxidant stress response elements (AREs) on their promoters, thereby activating the expression of a wide range of cytoprotective genes. The high or low expression of Nrf2 in astrocytes plays a decisive role in the ability of the entire nervous system to resist oxidative damage, which is accompanied by a functional deficit of Nrf2 in the mouse MPTP model. A variety of kinases can cause Nrf2 activation, including PKC, protein kinase CK2, PI3K, INK, ERK, etc., and thus phosphorylation is critical for regulating Nrf2-dependent gene expression. In eukaryotic cells, microtubules, microfilaments and intermediate filaments (IFs) together form the cytoskeletal structure. In the nervous system, IFs are mainly found in neurons and astrocytes. IFs in astrocytes include nestin, waveform protein, and connexin, and glial fibrillary acid protein (GFAP) is the predominant IF. In a variety of central nervous system (CNS) disorders, astrocytes are overly enlarged in size, and are changed from normotrophic astrocytes to activated astrocytes. In this process, the expression of IFs, especially GFAP, is increased, and the expression of GFAP in the hypothalamus is significantly increased in PD patients compared with normal subjects, suggesting that GFAP, a component of the astrocyte cytoskeleton, plays an important role in CNS diseases. In summary, the selective toxic effects of environmental toxins on nigrostriatal dopamine neurons involve complex mechanisms such as activation of the NF-κB signaling pathway, phosphorylation of proteins, and cytoskeletal alterations.

JWA gene (also known as ARL6IP5) is an environmental response gene discovered and cloned from retinoic acid-induced human bronchial epithelial (HBE) cell differentiation model by Zhou Jianwei et al. The encoded protein is a cytoskeletal-binding protein, which functions in regulating the cell differentiation, response to oxidative stress, DNA repair, etc. in normal cells. Using a Drosophila model with the JWA gene knocked out, it is found that Drosophila with defective JWA expression is less likely to tolerate repeated exposure to ethanol. A rat and cell model using antisense nucleic acid to inhibit JWA expression shows that JWA maintains the stability of the opiate receptor DOR through the ubiquitin proteasome pathway, and thus has a direct regulatory effect on morphine dependence in rats. JWA is involved in the regulation of signaling pathways such as NF-κB and MAPK, and it has been demonstrated that JWA participates in the regulation of cell senescence through the modulation of the activity of the NF-κB transcription factor on a cellular level, which is based on the mechanism that JWA is involved in cellular aging through the ubiquitin proteasome pathway. The mechanism is that JWA regulates the degradation of IKKβ through the ubiquitin proteasome pathway and inhibits the entry of p65 into the nucleus. Under oxidative stress, H₂O₂ induces the binding of NFI to the CCAAT element in the proximal promoter region of JWA, which activates the expression of JWA in response to oxidative stress. JWA regulates the expression of nuclear factor E2F1 and XRCC1 through the MAPK signaling pathway to enhance the repair ability of DNA damage.

In a study of astrocyte of A129 mice with JWA knockout, it is found that JWA knockout of astrocyte is extremely sensitive to MPTP, suggesting that JWA in astrocytes is a key molecule in the central nervous system to protect against stimulation by external toxicants. JWA inhibits paraquat-generated oxidative stress through MAPK and PI3K signaling pathways and activates GSH and Nrf2, effectively antagonizing paraquat-induced dopamine neuronal damage in mice (see FIG. 1A). To further investigate the role of astrocyte JWA in the development of PD, a chronic model of MPTP is constructed by using astrocyte of A57 mice with JWA knockout, and the molecular basis of astrocyte JWA affecting DA neuronal survival is explored in depth. The deletion of astrocyte JWA gene can increase the sensitivity of mice to MPTP and paraquat. In the mechanistic study, it is found for the first time that JWA gene could activate the downstream CREB transcription factors through both MAPK/ERK and PI3K/Akt signaling pathways to regulate the downstream GLT-1, and ultimately cause the alteration of extracellular interstitial glutamate content (see FIG. 1B). In addition, JWA also downregulates IKKβ thereby inhibiting the NFκB signaling pathway-mediated neuroinflammation on PD (see FIG. 1C). These early research results lay the foundation for realizing PD therapies targeting astrocytes JWA.

The peptide JP1, which is screened based on the functional fragments of JWA, enters the intracellular compartment after targeting the highly expressed integrin αVβ3 through its linked RGD sequence, negatively regulates the nuclear transcription factor SP1, and down-regulates the expression of αVβ3, which effectively inhibits the growth and metastasis of melanoma in mice. In addition, the peptide can cross the blood-brain barrier.

Although JP1 can target the highly expressed integrin αVβ3 on the surface of melanoma after connecting to the RGD sequence, it is unclear whether JP1-targeted peptides can be used to treat central nervous system diseases such as Parkinson's disease, and further research is needed.

### SUMMARY

To solve the aforesaid problems, one objective of the disclosure is provide an application of a JWA peptide in the preparation of anti-Parkinson's disease drugs, which can regulate the proliferation and activation of astrocytes/microglia in the brain tissue, so as to effectively inhibit the over-activation of astrocytes/microglia, and significantly increase the antagonism level of dopamine neuron against excitotoxicity, and reduce neuron death, thus providing a new clinical treatment of Parkinson's disease.

The disclosure provides a method for preventing or treating Parkinson's disease comprising administering a patient in need thereof a pharmaceutical composition comprising a polypeptide, the peptide having an amino acid sequence I or II:

I: FPGSDRF (SEQ ID NO: 1)-Z;
II: X-FPGSDRF (SEQ ID NO: 1)-Z;

S represents phosphorylated serine; X and Z independently represents an amino acid or an amino acid sequence;
X is selected from F, (R)₉ (SEQ ID NO: 2), (R)₉-F (SEQ ID NO: 3), 6-aminohexanoic acid, 6-aminohexanoic acid-F, 6-aminohexanoic acid-(R)₉ (SEQ ID NO: 2), 6-aminohexanoic acid-(R)₉-F (SEQ ID NO: 3); and
Z is selected from (G)ₙ-RGD or A-(G)ₙ-RGD (SEQ ID NO: 4), where n is an integer greater than or equal to 0, in the range of 0-10.

In a class of this embodiment, a symptom of the Parkinson's disease is excessive activation or proliferation of astrocytes or microglia in brain tissue.

In a class of this embodiment, a symptom of the Parkinson's disease is degeneration or loss of dopamine neurons in brain tissue.

In a class of this embodiment, a symptom of the Parkinson's disease is weakened vitality of hippocampal neurons or glial cells due to cytotoxic effects.

In a class of this embodiment, a symptom of the Parkinson's disease is increased expression of inflammasomes and increased apoptosis in hippocampal neurons or glial cells.

In a class of this embodiment, a symptom of the Parkinson's disease is increased membrane potential and elevated intracellular levels of reactive oxygen due to damage of cellular mitochondria in hippocampal neuronal cells or neuroglial cells.

In a class of this embodiment, the polypeptide comprises an acetylated N-terminal and an amidated C-terminal.

In a class of this embodiment, the polypeptide has an amino acid sequence of FPGSDRF-RGD, and serine (S) of the amino acid sequence of FPGSDRF-RGD is phosphorylated.

In a class of this embodiment, the pharmaceutical composition further comprises a pharmaceutically acceptable excipient.

The polypeptide involved in the disclosure is part of Chinese Patent Application No. CN201310178099. X. The polypeptides have therapeutic effects on Parkinson's disease, and can directly reach astrocytes/microglia in brain tissue through the blood-brain and other barriers by targeting integrin molecules and enter the cells to regulate the proliferation and activation, etc., effectively inhibit the over-activation of astrocytes/microglia, significantly increase the antagonism level of dopamine neuron against excitotoxicity, and reduce neuron death. Therefore, these peptides can be used as candidate molecules for the treatment or prevention of Parkinson's disease, or specific symptoms or signs of Parkinson's disease, for the preparation of corresponding drugs, and have good prospects for application.

### BRIEF DESCRIPTION OF THE DRAWINGS

FIG. 1A shows JWA inhibits paraquat-generated oxidative stress through MAPK and PI3K signaling pathways; FIG. 1B shows JWA inhibits paraquat-generated oxidative stress through MAPK and PI3K signaling pathways; FIG. 1C shows JWA downregulates IKKβ thereby inhibiting the NFκB signaling pathway-mediated neuroinflammation on PD;

FIG. 2 shows a graph of the results of analyzing JWA expression levels in whole blood samples from early stage Parkinson's disease patients and healthy control population in Example 1 of the disclosure; the Ctrl group is a healthy control population, and the PD group is early stage Parkinson's disease patients;

FIG. 3 shows a graph of the results of the analysis of the relationship between JWA and NF-κB expression levels in whole blood samples from patients with early Parkinson's disease in Example 2 of the disclosure;

FIG. 4 shows a graph of the results of the analysis of the expression levels of JWA and TH in brain tissues of Parkinson's disease patients in Example 3 of the disclosure;

FIG. 5 shows graph of the results of TH and JWA protein expression levels in brain tissue of wild-type C57BL/6 mice of different ages in Example 4 of the disclosure;

FIG. 6 shows a graph of the results of TH and JWA protein levels in striatal tissue of wild-type C57BL/6 mice of different ages in Example 5 of the disclosure;

FIG. 7 shows a design diagram of JP1 treatment of the mouse Parkinson's disease model induced MPTP in Example 6 of the disclosure; the diagram shows the scheme of MPTP modeling time and JP1 intervention time, as well as the administration treatment, where MPTP is administered as a subcutaneous injection and JP1 is administered as a tail vein injection, both once a day; JP1 is administered 1 day earlier than MPTP, and MPTP is administered 2 hours after JP1;

FIG. 8 shows a graph of the results of JP1 reversing MPTP-induced exploratory behavioral abnormalities (open field test) in mice with PD in Example 7 of the disclosure; the right pictures, from left to right, represent the solvent control group, the solvent+JP1 group, the MPTP group, and the MPTP+JP1 group, respectively;

FIG. 9 shows a graph of the results of JP1 reversing MPTP-induced behavioral abnormalities in mice (pole test and rotarod test) in Example 8 of the disclosure; the right pictures, from left to right, represent the solvent control group, the solvent+JP1 group, the MPTP group, and the MPTP+JP1 group, respectively;

FIG. 10 shows a graph of the results of JP1 antagonizing MPTP-induced dopamine neuron deletion (anti-TH IHC staining) in PD mice in Example 9 of the disclosure; the right pictures, from left to right, represent solvent control group, solvent+JP1 group, MPTP group, MPTP+JP1 group, respectively;

FIG. 11 shows a graph of the results of JP1 antagonizing MPTP-induced dopamine neuron deletion (Nichols staining) in PD mice in Example 10 of the disclosure; the right pictures, from left to right, represent the solvent control group, solvent+JP1 group, MPTP group, MPTP+JP1 group, respectively;

FIG. 12 shows a graph of the results of JP1 antagonizing MPTP-induced astrocyte proliferation (anti-GFAP IHC staining) in PD mice in Example 11 of the disclosure; the right pictures, from left to right, represent solvent control group, solvent+JP1 group, MPTP group, MPTP+JP1 group, respectively;

FIG. 13 shows a graph of the results of JP1 antagonizing MPTP-induced astrocyte proliferation (anti-Iba1 IHC staining) in PD mice in Example 12 of the disclosure; the right pictures, from left to right, represent solvent control group, solvent+JP1 group, MPTP group, MPTP+JP1 group, respectively;

FIGS. 14A-14D show cell viability of JP1 antagonizing rotenone (Rot) toxicity to the mouse hippocampal neuronal cell line HT-22 and the human neuroblastoma cell line SH-SYSY in Example 13 of the disclosure; FIG. 14A and FIG. 14B show cell viability after Rot treatment alone or Rot and JP1 co-treatments of HT-22 cells for 24 and 48 hours, respectively; FIG. 14C and FIG. 14D are cell viability after Rot treatment alone or Rot and JP1 co-treatments of SH-SYSY cells for 24 and 48 hours, respectively;

FIG. 15 is the results of JP1 antagonizing the abnormal expression of inflammatory vesicles (NLRP3), and apoptosis-related molecules (PARP1, Cleaved caspase1, 3, and 9) in HT-22 and SH-SYSY cells caused by Rot in Example 14 of the disclosure; and

FIGS. 16 and 17 are the results of JP1 antagonizing mitochondrial damage (mitochondrial membrane potential JC1) and oxidative stress (reactive oxygen species ROS) in HT-22 and SH-SYSY cells caused by rotenone (Rot) in Example 15 of the disclosure, respectively.

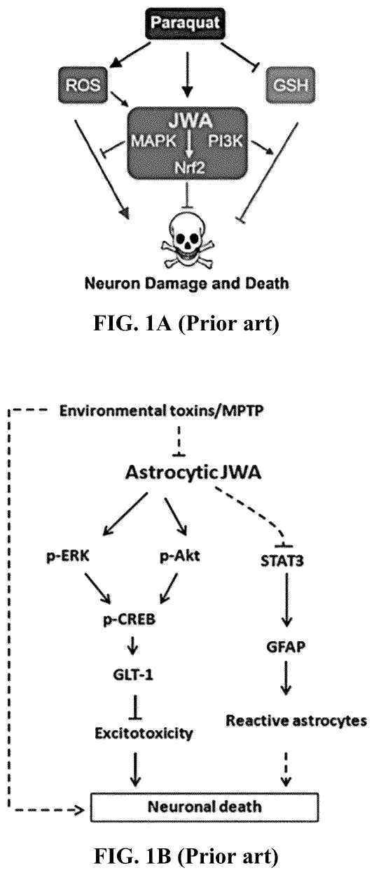

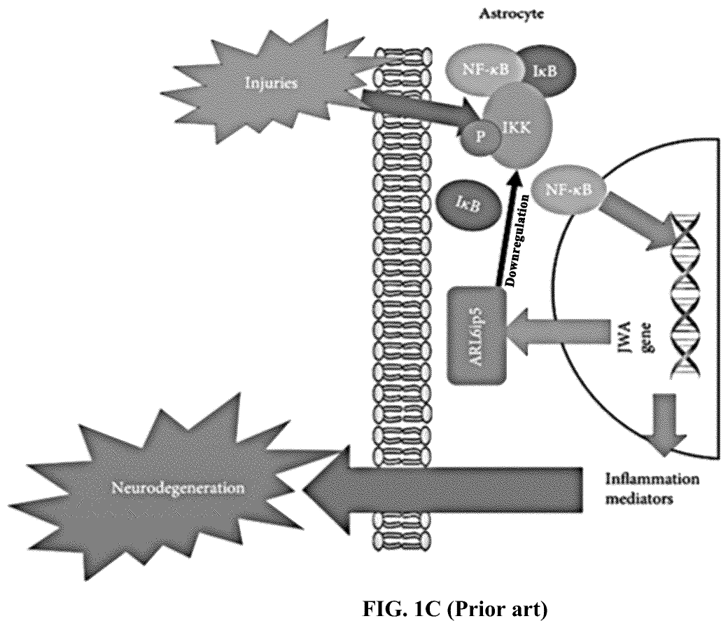

### DETAILED DESCRIPTION

To further illustrate the disclosure, embodiments detailing a method for preventing or treating Parkinson's disease are described below. It should be noted that the following embodiments are intended to describe and not to limit the disclosure.

### Example 1

In this example, the JWA expression level of whole blood samples from early stage Parkinson's disease patients and healthy population was analyzed according to the data from international public databases.

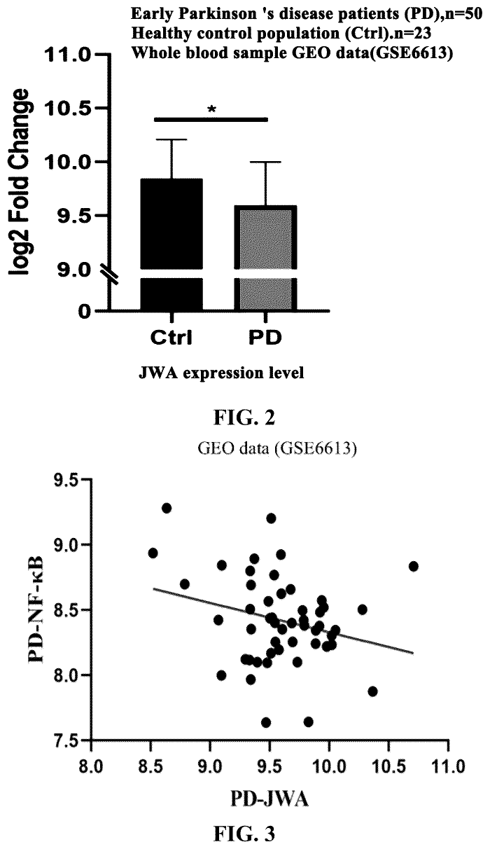

The results of the analysis are shown in FIG. 2: by analyzing the JWA expression levels of blood samples from 50 early stage Parkinson's disease patients and 23 healthy populations, the results show that the JWA expression levels in the blood of early stage PD patients are significantly lower compared to healthy populations, which provides evidence that JWA is a molecule that is disturbed in the blood cells from early stage PD patients.

### Example 2

In this example, the relationship between JWA and NF-κB expression levels in whole blood samples from patients with Parkinson's disease in early stages was analyzed based on data from international public databases.

The analysis results are shown in FIG. 3: by analyzing the relationship between the expression level of JWA and the expression level of the inflammatory factor NF-κB in whole blood samples from 50 patients with PD, the results showed that the expression level of JWA in patients with PD was negatively correlated with NF-κB.

### Example 3

In this example, the JWA and tyrosine hydroxylase (TH) expression levels of the Parkinson's disease stem cells were analyzed based on data from international public databases.

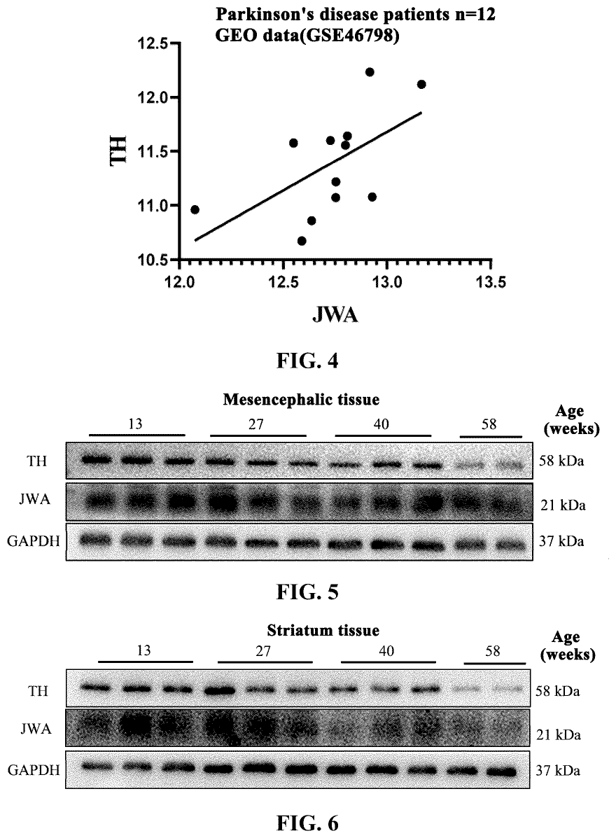

The results of the analysis are shown in FIG. 4: data on JWA and TH expression levels obtained from 12 PD patient stem cell models were analyzed, and the results showed a significant positive correlation between JWA expression levels and TH expression levels in PD stem cells.

### Example 4

The example is to detect the protein expression level of TH and JWA in the midbrain tissue of wild-type C57BL/6 mice of different ages.

The results are shown in FIG. 5: Western blotting analysis of normal wild-type C57BL/6 mouse midbrain tissues of different ages showed that with the growth of age of mice, the protein expression levels of tyrosine hydroxylase (TH) and JWA in the midbrain tissues of mice presented a gradual decreasing trend, and the two molecules changed in a consistent pattern. The decrease in the expression level of TH indicates the gradual decrease of dopamine neurons, while the decrease in the expression level of JWA suggests the gradual weakening of its anti-oxidative stress, anti-inflammatory, and antagonistic function against the development of PD.

### Example 5

The example is to detect the protein levels of TH and JWA in striatal tissues of wild-type C57BL/6 mice of different ages.

The results are shown in FIG. 6: Western blotting analysis of striatal tissues of normal wild-type C57BL/6 mice of different ages showed that the protein expression of TH and JWA in striatal tissues of mice showed a gradual decrease trend with the age of mice, and the change rule of the expression of the two molecules was consistent.

### Example 6

The example is a protocol designed to validate the effect of JP1 for the treatment of MPTP-induced Parkinson's disease in mice. Note: The sequence of JP1 is FPGSDRF-RGD (SEQ ID NO: 27), where the amino acid S is phosphorylated.

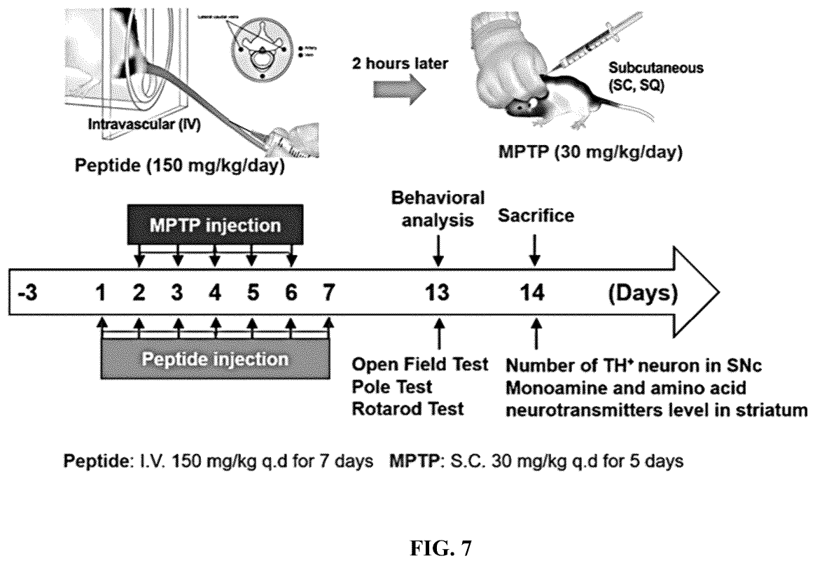

As shown in FIG. 7, the design scheme to validate the anti-PD effect of JP1 included dividing C57BL/6 mice into four groups: solvent control group, solvent+JP1 group, MPTP group, and MPTP+JP1 group.

For the MPTP+JP1 group: JP1 was administered via tail vein injection at a dose of 150 mg/kg once a day for 7 consecutive days; on the next day following the administration of JP1, MPTP, a toxicant for the mouse PD model, was administered through dorsal subcutaneous injection at a dose of 30 mg/kg once a day and 2 h after the administration of JP1, and probenecid (250 mg/kg) was administered intraperitoneally with a microinjector 1 h after the MPTP injection (ready-to-use). Both MPTP and probenecid were administered for 5 consecutive days.

For the MPTP group, MPTP (30 mg/kg) was administered by dorsal subcutaneous injection, and probenecid (250 mg/kg) was administered by intraperitoneal injection (ready-to-use) using a microinjector 1 h after MPTP injection. The drug was administered for 5 consecutive days.

For solvent+JP1 group: JP1 was administered via tail vein injection in mice at a dose of 150 mg/kg once daily for 7 consecutive days.

For the solvent control group: the mice were administered according to the operations in the MPTP group, replacing MPTP with sterile saline in equal amounts, and the rest were the same.

The starting point of timing was standardized for all groups, with the 1st administration of JP1 as day 1 and the 1st administration of MPTP as day 2. Behavioral change characteristics of the model mice were evaluated on Day 13 after the start of the modeling, and the modeling was ended on day 14. The brain tissues were removed, and brain slices were prepared for the analysis of PD-related molecular marker assays and assessment of treatment effects.

### Example 7

The example is to assess the therapeutic effect of JP1 to reverse MPTP-induced exploratory behavioral abnormalities in mice with PD (open field test).

The open field test is used to validate behavioral changes in MPTP-treated PD mice in an open field. The assay assessment time point is Day 13 of the initiation of dosing in the design scheme of Example 6. It was used to detect spontaneous activity behavior and exploratory behavior in mice. PD model mice often exhibit reduced spontaneous activity behavior and inactive exploratory behavior, and are insensitive to the fresh external environment.

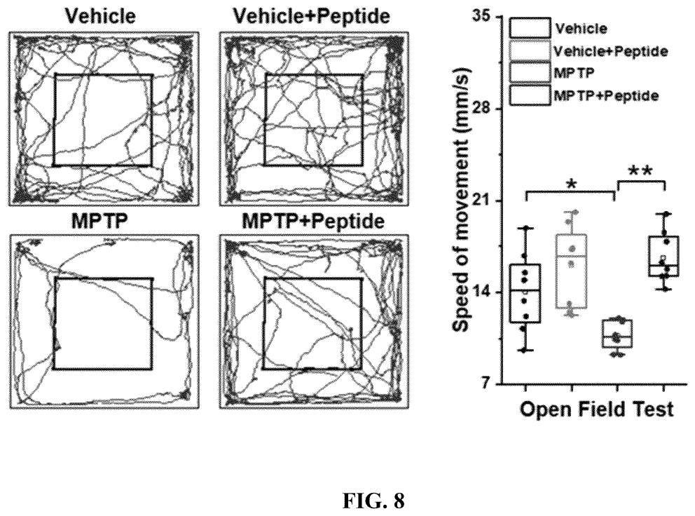

The results are shown in FIG. 8: compared with the solvent control group, the solvent+JP1 group showed that the JP1 treatment increased spontaneous activity and exploratory behavior; the MPTP group showed that MPTP-treated mice showed a significant decrease in spontaneous activity and exploratory behavior to the center of the field (P<0.05); the MPTP+JP1 group showed that the JP1 intervention significantly reversed the decrease in spontaneous activity and exploratory behavior caused by the MPTP treatment (P<0.01).

### Example 8

The example is to evaluate the therapeutic effect of JP1 in reversing MPTP-induced behavioral abnormalities in mice (pole test and rotarod tests).

The pole test and rotarod test is to present behavioral indicators used to validate neural coordination in MPTP-treated PD mice. The test assessment time point is Day 13 of initiation of dosing in the design scheme of Example 6. The pole test was used to assess basal ganglia-related motor deficits in mice, and the rotarod test was used to test the coordination function of the central nervous system of mice. PD can be characterized by prolonged pole test time and reduced rotarod coordination, and shorter falling time of rotarod.

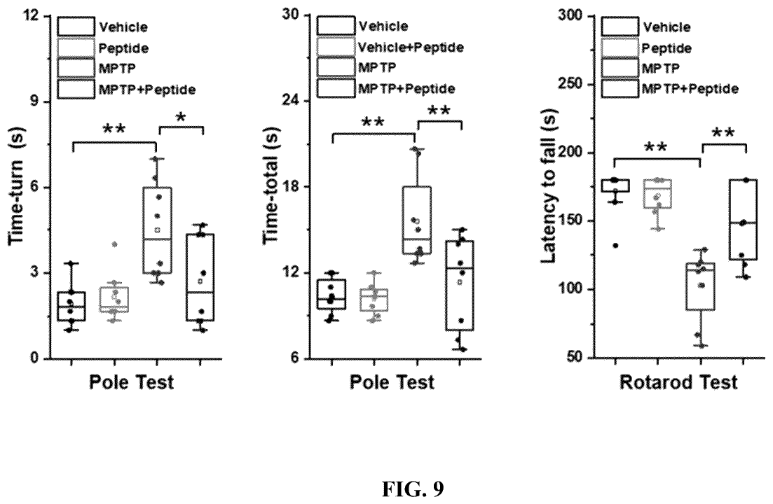

The results are shown in FIG. 9: compared with the solvent control group, the solvent+JP1 group showed that JP1 treatment alone did not have a significant influence on rod-climbing time and falling time from the rotating rod in mice; the MPTP group showed that MPTP-treated PD model mice, on the other hand, had a significantly longer rod-climbing time (P<0.01) and a significantly shorter falling time from the rotating rod; and the MPTP+JP1 group demonstrated that the JP1 intervention could significantly improve MPTP-induced changes in rod climbing and rotarod behaviors in mice (P<0.01).

### Example 9

The example was used to assess the therapeutic effect of JP1 antagonizing MPTP-induced dopamine neuron deficits in PD mice (anti-TH IHC staining).

Changes in TH expression were used to validate dopamine neurons in dense areas of the midbrain substantia nigra in MPTP-treated PD mice. The assay was evaluated on Day 14 of the design scheme of Example 6. TH is a monooxygenase, which is a rate-limiting enzyme that catalyzes the first step in the synthesis of L-Dopamine (DA) by the organism itself, and the level of TH expression actually reflects the number of dopamine neurons and the level of the transporter in the brain, which is often significantly reduced in PD.

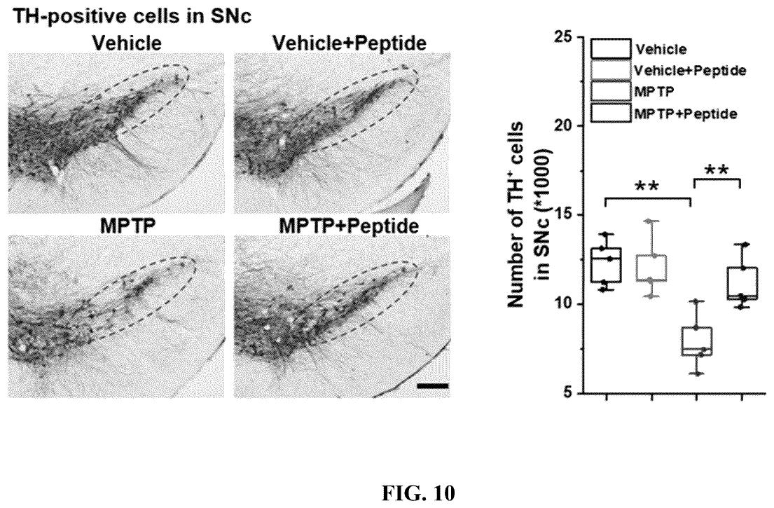

The results are shown in FIG. 10: compared with the solvent control group, the solvent+JP1 group showed that JP1 treatment alone had no significant effect on the TH expression level; the MPTP group showed that the TH expression level in the MPTP-treated group was significantly decreased (P<0.01); and the MPTP+JP1 group showed that the JP1 intervention significantly improved the TH level (P<0.01).

### Example 10

The example is to assess the therapeutic effect of JP1 antagonizing MPTP-induced dopamine neuron deficits in PD mice (Nissl staining).

Nissl granules in neuronal cytoplasm were stained to assess the number of midbrain dopamine neurons in MPTP-induced PD model mice. The assay assessment time point was Day 14 of initiation of dosing in the design scheme of Example 6. Nissl bodies are sites of neuronal protein synthesis, and the number of Nissl bodies decreases when neurons are stimulated and injured. Therefore, high staining results are often used as an observation of neuronal damage.

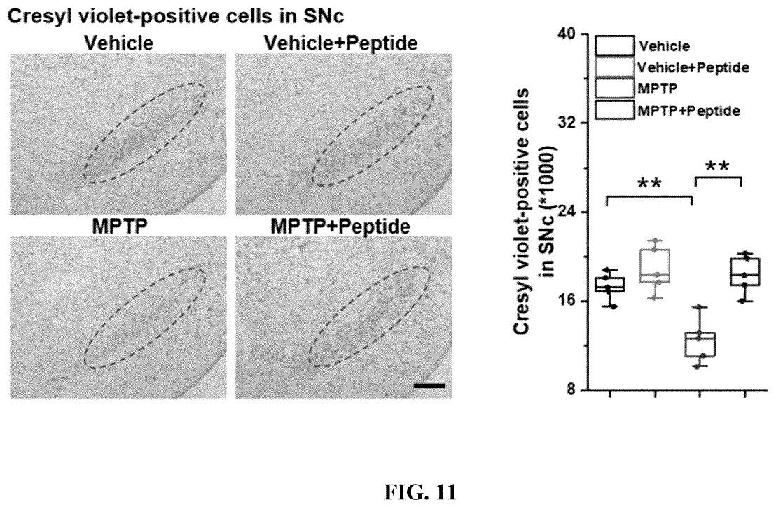

The results are shown in FIG. 11: compared with the solvent control group, the solvent+JP1 group showed that JP1 treatment alone had no significant effect on the number of Nissl bodies in the midbrain multiply pressed neurons; the MPTP group showed that MPTP-treated PD mice, on the other hand, showed a significant reduction in Nissl bodies (P<0.01); the MPTP+JP1 group showed that JP1 intervention significantly reversed the MPTP-induced reduction of Nissl bodies of dopamine neuron (P<0.01).

### Example 11

The example is to assess the therapeutic effect of JP1 antagonizing MPTP-induced astrocyte proliferation in PD mice (anti-GFAP IHC staining).

A PD model was built and characterized to assess the expression level of the glial fibrillary acidic protein (GFAP) for brain astrocyte activation and proliferation. The assay assessment time point was Day 14 of the design scheme of Example 6. GFAP, a glial-derived fibrillary acidic protein, is a molecular marker specific for astrocytes, and its high expression level reflects the number and activation of brain astrocytes.

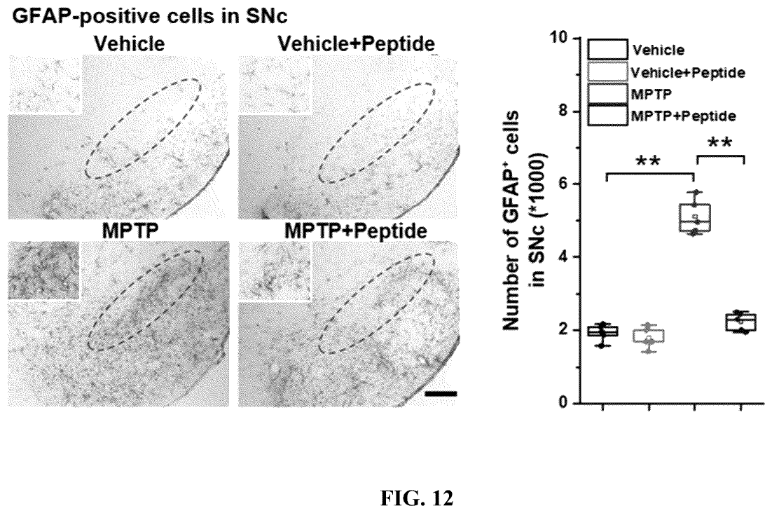

The results are shown in FIG. 12: compared with the solvent control group, the solvent+JP1 group showed that JP1 treatment alone had no significant effect on the GFAP expression level in brain astrocytes; the MPTP group showed that a significant increase in the GFAP expression level in MPTP-treated PD mice (P<0.01); and the MPTP+JP1 group showed that the JP1 intervention significantly reversed the MPTP-induced increase in GFAP (P<0.01).

### Example 12

The example is to evaluate the therapeutic effect of JP1 on MPTP induced microglial proliferation in PD mice (anti-Iba1 IHC staining).

The expression levels of molecular markers are used to validate the activation of microglia in MPTP induced PD models. The testing and evaluation time point is the 14th day from the start of administration in the implementation design plan. Microglia is the main immune inflammatory cells in brain tissue. Calcium ions are one of the most important signaling molecules in all known cells, including central nervous system (CNS) cells. Calcium ions exert signaling activity by binding to various calcium binding proteins, many of which are classified into a large protein family, the EF chiral protein family. Calcium binding protein-1 (Iba1) is a 17 kDa EF chiral protein expressed in microglia and upregulated during the activation process of these cells.

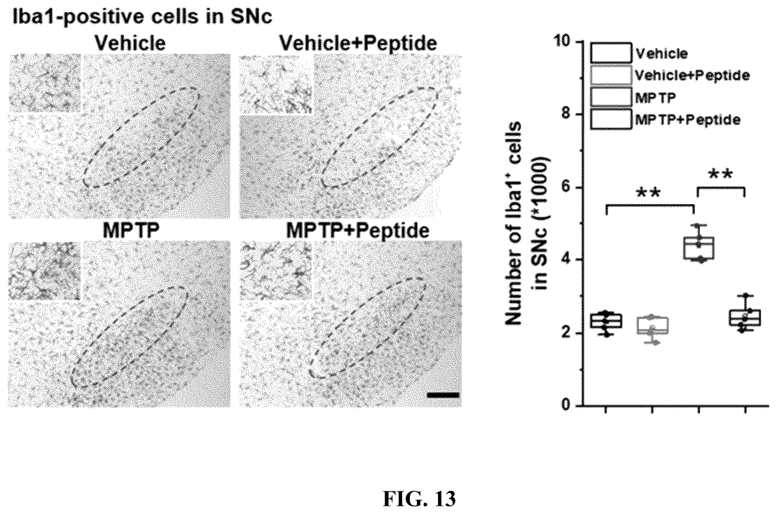

As shown in FIG. 13, compared with the solvent control group, the solvent+JP1 group showed that JP1 alone had no significant effect on the expression level of Iba-1 in the dense area of the substantia nigra of the midbrain; the MPTP group showed a significant increase in Iba-1 expression in MPTP treated PD mice (P<0.01); the MPTP+JP1 group showed that JP1 intervention could reverse the increase in Iba-1 caused by MPTP (P<0.01).

### Example 13

The example is to evaluate the protective effects of JP1 on the viability of mouse hippocampal neuronal cells HT-22 and human glioblastoma cells (SH-SYSY) induced by Rotenone (Rot) (CCK8 assay).

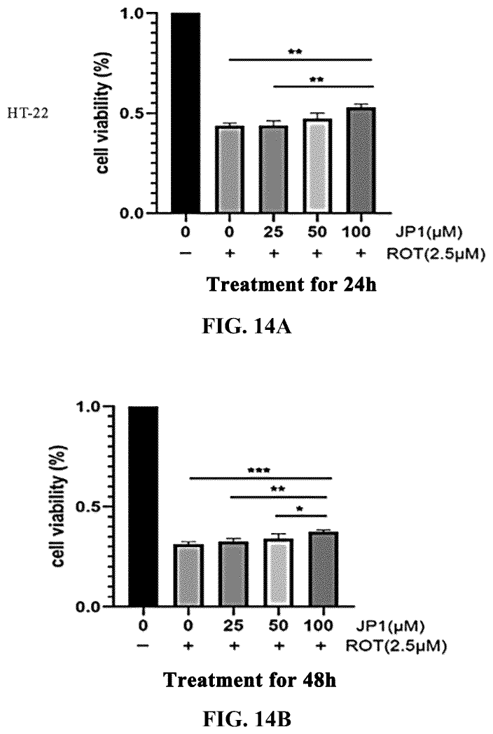

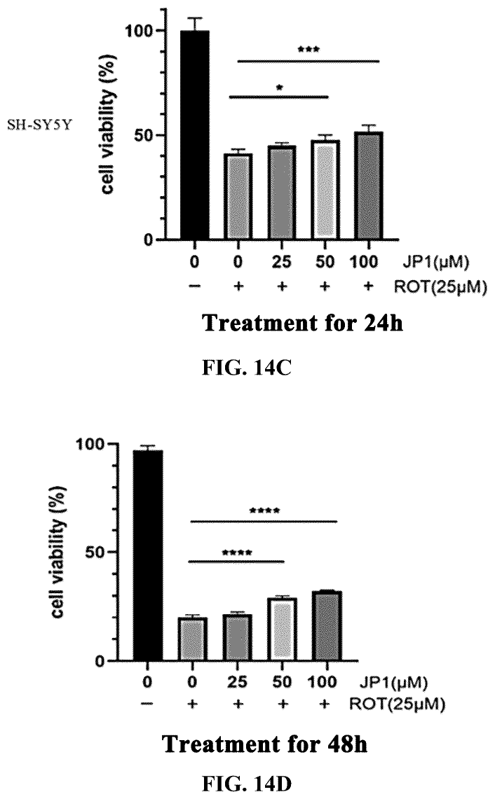

As shown in FIGS. 14A-14D, the cell viability of HT-22 cells treated with Rot (2.5 μM) alone or with Rot (2.5 μM) and JP1 (25, 50, 100 μM) for 24 and 48 hours showed that compared with the solvent control group, the viability of the two cells significantly decreased after 24 hours of Rot treatment, and the viability of the two cells decreased more significantly after 48 hours of Rot treatment compared to the 24-hour group. The results of the combined treatment group of JP1 and Rot showed that JP1 had a significant protective effect on the cytotoxicity of Rot cells, and exhibited a dose-dependent effect.

### Example 14

The example is to assess the antagonistic effect of JP1 on the inflammatory apoptosis in HT-22 neuronal cells and SH-SYSY glial cells caused by rotenone (Rot), respectively.

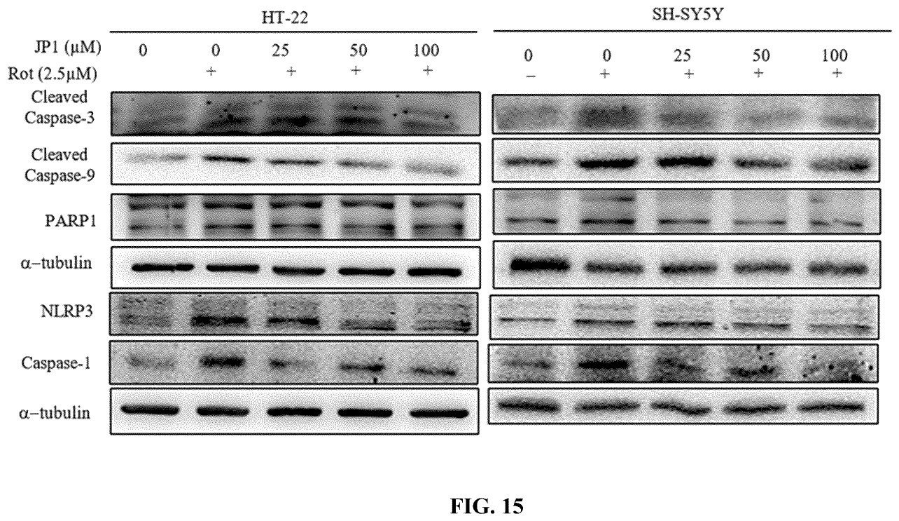

The results are shown in FIG. 15, both cells were treated with control solvent, Rot alone or ROT+JP1 combination for 48 h. The result showed that Rot treatment significantly activated and increased the expression levels of inflammatory vesicle NLRP3 as well as apoptosis-related molecules (PARP1, Cleaved caspase1, 3, 9) in both cells compared to solvent control group. In contrast, the expression levels of both inflammatory vesicles and apoptosis-related molecules were suppressed in the combination of Rot and JP1 treatment group in a dose-dependent manner.

### Example 15

The example is to evaluate the antagonism of JP1 against Rotenone induced mitochondrial damage (mitochondrial membrane potential JC1) and its oxidative stress mechanism (reactive oxygen species ROS) in HT-22 and SH-SYSY cells.

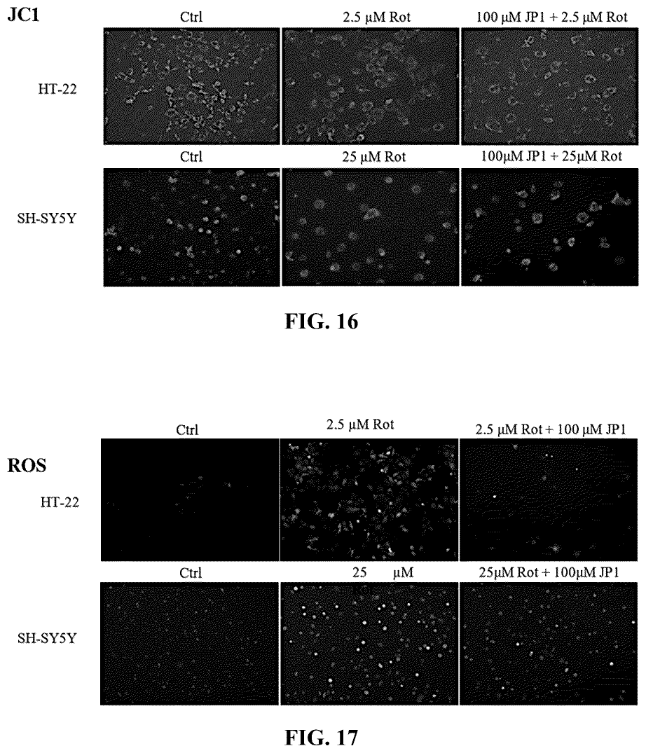

As shown in FIG. 16 and FIG. 17, both cells were treated with control solvent, Rot alone or ROT+JP1 combination for 48 h. The result showed that Rot treatment (2.5 μM, 48 h) resulted in severe mitochondrial membrane damage and a significant increase in intracellular oxidative stress (ROS) in both cells, while Rot-JP1 (100 μM, 48 h) co-treatment resulted in a significant reduction of mitochondrial membrane damage and intracellular oxidative stress in both cells.

### Example 16

The example is to verify the anti-Parkinson's disease effect of JWA peptides other than JP1.

The example uses the JWA peptides shown in the following table for tests according to Examples 6 to 12. The amino acid S of each JWA peptide is phosphorylated.

| No. | Sequence | No. | Sequence | No. | Sequence |
|---|---|---|---|---|---|
| JP2 | FPGSDRF-G- | JP3 | FPGSDRF-(G)₄- | JP4 | FPGSDRF-(G)₁₀- |
| | RGD (SEQ ID | | RGD (SEQ ID | | RGD (SEQ ID |
| | NO: 5) | | NO: 6) | | NO: 7) |
| JP5 | FPGSDRF-A- | JP6 | FPGSDRF-A-G- | JP7 | FPGSDRF-A- |
| | RGD (SEQ ID | | RGD (SEQ ID | | (G)₄-RGD (SEQ |
| | NO: 8) | | NO: 9) | | ID NO: 10) |
| JP8 | FPGSDRF-A- | JP9 | FFPGSDRF-RGD | JP10 | FFPGSDRF-G- |
| | (G)₁₀-RGD (SEQ | | (SEQ ID NO: 12) | | RGD (SEQ ID |
| | ID NO: 11) | | | | NO: 13) |
| JP11 | FFPGSDRF-(G)₄- | JP12 | FFPGSDRF-(G)₁₀- | JP13 | FFPGSDRF-A- |
| | RGD (SEQ ID | | RGD (SEQ ID | | RGD (SEQ ID |
| | NO: 14) | | NO: 15) | | NO: 16) |
| JP14 | FFPGSDRF-A-G- | JP15 | FFPGSDRF-A- | JP16 | (R)₉-FPGSDRF- |
| | RGD (SEQ ID | | (G)₁₀-RGD (SEQ | | RGD (SEQ ID |
| | NO: 17) | | ID NO: 18) | | NO: 19) |
| JP17 | (R)₉-FPGSDRF- | JP18 | (R)₉-FPGSDRF- | JP19 | (R)₉-FPGSDRF- |
| | (G)₁₀-RGD (SEQ | | A-RGD (SEQ ID | | A-(G)₁₀-RGD |
| | ID NO: 20) | | NO: 21) | | (SEQ ID NO: 22) |
| JP20 | (R)₉-F- | JP21 | (R)₉-F-FPGSDRF- | JP22 | (R)₉-F- |
| | FPGSDRF-RGD | | (G)₁₀-RGD (SEQ | | FPGSDRF-A- |
| | (SEQ ID NO: 23) | | ID NO: 24) | | RGD (SEQ ID |
| | | | | | NO: 25) |
| JP23 | (R)₉-F- | JP24 | 6-aminocaproic | JP25 | 6-aminocaproic |
| | FPGSDRF-A- | | acid-FPGSDRF- | | acid-FPGSDRF- |
| | (G)₁₀-RGD (SEQ | | RGD (SEQ ID | | (G)₁₀-RGD (SEQ |
| | ID NO: 26) | | NO: 27) | | ID NO: 7) |
| JP26 | 6-aminocaproic | JP27 | 6-aminocaproic | JP28 | 6-aminocaproic |
| | acid-FPGSDRF- | | acid-FPGSDRF-A- | | acid-F-FPGSDRF- |
| | A-RGD (SEQ ID | | (G)₁₀-RGD (SEQ | | RGD (SEQ ID |
| | NO: 8) | | ID NO: 11) | | NO: 12) |
| JP29 | 6-aminocaproic | JP30 | 6-aminocaproic | JP31 | 6-aminocaproic |
| | acid-F-FPGSDRF- | | acid-F-FPGSDRF- | | acid-F-FPGSDRF- |
| | (G)₁₀-RGD (SEQ | | A-RGD (SEQ ID | | A-(G)₁₀-RGD |
| | ID NO: 15) | | NO: 16) | | (SEQ ID NO: 18) |
| JP32 | 6-aminocaproic | JP33 | 6-aminocaproic | JP34 | 6-aminocaproic |
| | acid-(R)₉- | | acid-(R)₉- | | acid-(R)₉- |
| | FPGSDRF-RGD | | FPGSDRF-(G)₁₀- | | FPGSDRF-A- |
| | (SEQ ID NO: 19) | | RGD (SEQ ID | | RGD (SEQ ID |
| | | | NO: 20) | | NO: 21) |
| JP35 | 6-aminocaproic | JP36 | 6-aminocaproic | JP37 | 6-aminocaproic |
| | acid-(R)₉- | | acid-(R)₉-F- | | acid-(R)₉-F- |
| | FPGSDRF-A- | | FPGSDRF-RGD | | FPGSDRF-(G)₁₀- |
| | (G)₁₀-RGD (SEQ | | (SEQ ID NO: 23) | | RGD (SEQ ID |
| | ID NO: 22) | | | | NO: 24) |
| JP38 | 6-aminocaproic | JP39 | 6-aminocaproic | | |
| | acid-(R)₉-F- | | acid-(R)₉-F- | | |
| | FPGSDRF-A- | | FPGSDRF-A- | | |
| | RGD (SEQ ID | | (G)₁₀-RGD (SEQ | | |
| | NO: 25) | | ID NO: 26) | | |

Due to space limitations, specific experimental data are not listed in the example. The obtained experimental data indicate that the test results of each JWA peptide according to Examples 6 to 12 are basically consistent with that of JP1.

### CONCLUSION

As can be seen from the above examples, the disclosure confirms the therapeutic effect of a series of JWA polypeptides represented by JP1 on MPTP-induced Parkinson's disease in a mouse model. These JWA peptides can directly reach astrocytes/microglia in brain tissue via targeting integrin molecules through the blood-brain and other barriers, and enter the cells to play the roles of regulating proliferation and activation, etc., and effectively inhibit the over-activation or proliferation of astrocytes/microglia, as well as the consequent death of dopamine neurons. These JWA peptides can significantly increase the antagonism level of dopamine neuron against excitatory neurotoxicity and reduce neuronal death. Reducing the loss of dopamine neurons can maintain the neurobehavioral homeostasis mediated by dopamine neurons. In addition, behavioral abnormalities in PD mice are significantly ameliorated through the intervention of these JWA peptides. Thus, these polypeptides may be promising as candidate molecules for use in the preparation of drugs targeting Parkinson's disease for the treatment or prevention of Parkinson's disease.

The materials, methods, experimental model conditions, etc. used in the above examples are shown below.

### 1. Main Reagents and Antibodies and Configuration

Main chemical reagents: domestic AR grade reagents comprising: sodium chloride, acrylamide, ammonium persulfate, acrylamide, N′,N′,N′,N′-tetramethylethylenediamine (TEMED), sodium bicarbonate, sodium dihydrogen phosphate, disodium hydrogen phosphate and sodium dodecyl sulfate (SDS). Immunohistochemical chromogenic reagent DAB (AR1000) was purchased from Wuhan Boshi Bioengineering Co. Ltd. MPTP, probenecid and polylysine were from Sigma-Aldrich, USA. Peptides (including JP1, etc.) were synthesized by GL Biochem (Shanghai) Co., Ltd. and Hybio Pharmaceutical Co., Ltd (Shenzhen, China) under standard GMP conditions, with purity >98%, water-soluble, and lyophilized powders were stored at −20° C. for a long time.

The primary antibody JWA monoclonal antibody was prepared by Beijing Jingtiancheng Biotechnology Co. Ltd. Primary antibodies: GLT-1, GLAST, p-P65 antibody (Abcam, Cambridge, UK); Iba-1 antibody (Proteintech, Chicago, IL, USA); TH antibody (Sigma-Aldrich St. Louis, MO, USA); GFAP (SAB Signalway Antibody, Maryland, USA).

Immunohistochemical reagents were prepared as follows: 4% paraformaldehyde (PFA): 4 g of PFA powders were weighed, fully dissolved in 100 mL of PBS solution, and filtered for use. 95% ethanol: 190 mL of anhydrous ethanol was measured using a measuring cylinder, and ddH₂O was added to a constant volume of 200 mL. 80% ethanol: 160 mL of anhydrous ethanol was measured using a measuring cylinder, and ddH₂O was added to a constant volume of 200 mL. 70% ethanol: 140 mL of anhydrous ethanol was measured using a measuring cylinder, and ddH₂O was added to a constant volume of 200 mL. PBST: 1 L ddH₂O+7 g Na₂HPO₄·12H₂O+0.5 g NaH₂PO₄·2H₂O+9 g NaCl were dissolved in 1000 mL of water.

Preparation of general anesthetics: mice: 2.5% chloral hydrate+5% ulatan, starting dose 0.1 mL/20 g, intraperitoneal administration 0.2 mL. Formulation: 10 mL NS+0.25 g chloral hydrate+0.5 g ulatan.

### 2. Cell and Animal Sources and PD Model Preparation

1) Cell culture and treatment: The human neuroblastoma cell line SH-SYSY and the mouse hippocampal neuron cell line HT-22 were cultured in DMEM medium containing 100 g/mL streptomycin, 100 U/mL penicillin, and 10% fetal bovine serum at 37° C. in a 5% CO₂ environment. The cells were treated with Rotenone (2.5 μM) and JWA targeting peptide JP1 (0, 25, 50, 100 μM) for 24 and 48 hours, respectively. Model cells were tested for cell viability using CCK8 assay, expression levels of inflammasome 3 (NLRP3) and apoptosis related proteins (PARP1, Cleaved caspase1, 3, 9) using Western Blot, and mitochondrial membrane potential (JC1) and intracellular reactive oxygen species (ROS) levels using immunofluorescence staining.

2) Mouse model of subacute Parkinson's disease: 3-4 months old C57BL/6 mice weighing 25-30 g were used, and all mice were kept in the Laboratory Animal Center of Nanjing Medical University (SPF environment). The mice were fed ad libitum with standard diet, indoor temperature was maintained at (24±2°) C, indoor humidity was 50-60%, and the room was well ventilated, with 12 h of light and 12 h of darkness per day in the Laboratory Animal Center of Nanjing Medical University (NJMU), and all the experiments were carried out in accordance with the Guidelines for Experimental Animal Research of NJMU, and approved by the Institutional Animal Care and Use Committee of NJMU.

Preparation of MPTP mouse PD model: MPTP storage solution (200 mg/kg) was prepared with sterile saline and stored in an ultra-low-temperature refrigerator at −80° C. When for use, the storage solution was melted on ice and diluted 10-fold in sterile saline. MPTP (30 mg/kg) was administered by dorsal subcutaneous injection, and probenecid (250 mg/kg) was administered intraperitoneally with a microinjector (ready to use) 1 h after the MPTP injection. The mice were administered for 5 consecutive days and were uniformly executed on the 7th day after the last administration. The tissues were taken on ice for the experiments. The control mice were treated with sterile saline and the same dose of probenecid. In the JP1 alone treatment group or the JP1 intervention group, the drug was diluted with sterile saline and injected intraperitoneally at the dose of 150 mg/kg once a day for 7 consecutive days. The model mice were treated with MPTP on the 2nd day after the injection of JP1, and the MPTP was given 2 hours after the JP1 administration. Probenecid (250 mg/kg, ready to use) was administered intraperitoneally with a micro syringe 1 h after MPTP injection, and both MPTP and probenecid were administered for 5 consecutive days. The last administration of JP1 was given on Day 7, and the behavioral assays were performed on the sixth day (i.e., Day 13 of the model) after the drug was stopped, and the mice were executed on the following day (i.e., day 14 of the model). Brain tissues were extracted and brain slices were prepared for evaluation of relevant molecular markers.

### 3. Cytotoxicity Assay:

Cells were inoculated in a 96-well plate at a density of 8000 cells/well for adherence for 24 hours. The culture medium was discarded and the cells were incubated for 24 and 48 hours with different concentrations of the drug. The original culture medium of the 96-well plate was discarded, 10 μL of CCK-8 reagent mixed with 90 μL of fresh culture medium were added to each well, and the cells were incubated for about 2 hours at 37° C. in a 5% CO2 incubator. The absorbance value was read by an enzyme immunoassay detector at 450 nm. The cell viability (3 parallel samples in each group, and repeat 3 times) was calculated.

### 4. Detection of Cellular Mitochondrial Membrane Potential and Intracellular Reactive Oxygen Species

According to the test requirements and the instructions of Cellular Mitochondrial Membrane Potential Assay and Intracellular Reactive Oxygen Species Assay Reagent (Biyuntian, Nanjing, China), the cells were treated and protected from light, loaded with DCFH-DA probe (the ratio of probe to culture medium volume was 1:1000) and incubated in a cell culture incubator at 37° C. for 20 min. The cells were washed three times with serum-free DMEM cell culture medium. Five visual fields were observed in each well under fluorescence inverted microscope, and the average was taken.

### 5. Mouse Behavioral Test

Behavioral training was conducted on the 2nd-3rd day after the last injection of poison. To avoid over-exertion of the mice, the training programs should be interspersed, and no training is required for the open field test. Pole Test: before the formal experiment, mice were trained to climb poles three times a day, with the mice resting in the rest time to avoid overexertion. At the beginning of the experiment, the mice were placed head up on the top of a pole (1 cm in diameter and 50 cm in height) and the time taken to climb from the top to the bottom of the pole was recorded. Rotarod Test: before the formal test, the mice were trained to rotate the rod at a constant speed (12 rpm) and the training time should not be too long (no more than 300 sec), and the speed of the rod should be accelerated (5-20 rpm) during the formal test, and the time the mice stayed on the rod was recorded. Open-Field Test: the mice were acclimatized to the open field for 15 min (the size of the open field was 20 cm×20 cm×15 cm), and an Open field software was used to record the total distance crawled by the mice within 10 min.

### 6. Preparation of Frozen Sections of Brain Tissue and Immunohistochemical Staining

6.1) Mice were anesthetized by intraperitoneal injection of pentobarbital, and after anesthesia, the left ventricle was perfused with 100 mL of saline and 100 mL of 4% paraformaldehyde. The brain tissues were carefully extracted after the cerebral shells were peeled off, fixed in 4% paraformaldehyde, and placed in a refrigerator at 4° C. overnight.

6.2) On day 2, the brain tissue was taken out from 4% paraformaldehyde and dehydrated in 20% sucrose solution; the sucrose solution was changed daily for 3 days, and then the brain tissue was taken out and dehydrated in 30% sucrose solution on a gradient basis for 3 days and changed daily for 3 days.

6.3) The brain tissue was embedded and sliced with a freezing microtome at a thickness of 25 m. The areas to be sliced were accurately identified according to the mouse brain atlas, with the striatum taken at intervals of 6 and the midbrain at intervals of 3. The slices were washed three times with PBS and finally collected in 1.5 mL EP tubes, to which were added PBS and glycerol (1:1) The slices were stored for a long period of time in a refrigerator at −20° C.

### 6.4) Experimental Steps of Brain Tissue Immunohistochemistry

a) The brain slices were rinsed with PBS and placed on a shaker 3 times, each for 15 min. 3% H₂O₂ was used to treat the slices for 15-20 min.

b) The slices were rinsed again with PBS and washed on a shaker 3 times for 15 min each, and then a 5% BSA blocking solution was prepared with 0.3% Triton PBS and the slices were blocked for 1 h at room temperature.

c) The primary antibody was incubated at room temperature for 1 h and at 4° C. overnight. The primary antibodies used in the disclosure were JWA, tyrosine hydroxylase, anti-glial-derived acidic protein and glutamate transporter protein.

d) After rinsing with 0.01 M PBS for 3×10 min, horseradish peroxidase-labeled secondary antibody (1:1000) was added and incubated at room temperature for 1 h. The brain slices were rinsed with PBS for 15 min each time for three times, and 3,3′-diaminobenzidine (DAB) was used for display for 15 min. The slices were dehydrated with gradient ethanol and sealed with neutral gum. The brain slices were photographed and counted with a stereomicroscope (Axiovert LSM510, Carl Zeiss Co.).

### 7. Western Blotting

### 7.1) Preparation of Midbrain and Striatal Tissue Protein

a) On the next day after the behavioral test, the mouse brain tissue was taken. Specifically, after the blood was taken from the eyeballs, the mice were executed by breaking the neck, and the brain tissue was carefully extracted from the brain shell of the mice to keep the brain tissue as intact as possible. The mouse brain tissue was symmetrically cut open to separate the midbrain, the striatum and the hippocampus.

The tissues were weighed and mixed with the protein cleavage liquid RIPA in the ratio of 1:8. The resulting mixture was allowed to cleavage in a refrigerator at 4° C. for 30 min. The supernatant was collected by centrifugation at 12000 g for 15 min at 4° C.

b) The protein concentration of striatal and midbrain tissues was determined using a BCA protein quantification method. According to the number of samples, the BCA working solution (A:B is 50:1) was prepared and mixed thoroughly, and 5 mg/mL bovine serum albumin (BSA) was used as the standard protein for control. 10 L of the original solution and 90 μL of NaCl were mixed. The preparation of the labeling curve included: stepwise adding the standard protein to a 96-well plate, adding NaCl supplemented to 20 μL; adding 1 μL of a sample to be tested into the 96-well plate, adding NaCl to 20 μL. 200 μL of BCA working solution was added to the wells of the labeling curve and the wells of the sample to be tested, and placed in an incubator at 37° C. for 30 min; the concentration of the protein was determined using an enzyme marker. The protein concentration was calculated according to the absorbance value and the standard curve.

c) 6×loading buffer in volume was added to the protein supernatant and the mixture was denatured in a metal bath at 100° C. for 5 min, and stored at −20° C. after sub-packaging.

### 7.2) Preparation of Cellular Protein

The culture fluid in the cell culture plate was discarded, and the remaining product was rinsed twice with pre-cooled PBS buffer or NaCl, and added to protein lysis solution, lysed in a refrigerator at 4° C. for 30 min, centrifuged at 12000 g for 15 min at 4° C. 1 μL of the protein sample was taken for the determination of protein concentration according to the above tissue protein concentration determination method, and the rest protein supernatant was added to 6×sample buffer in volume in a metal bath at 100° C.

### 7.3) Preparation of SDS-PAGE

a) Different concentrations of separation gel were selected according to different protein molecular mass. The larger the molecular weight, the lower the concentration of the separation gel. In the disclosure, the separation gel having a concentration of 12.5% was selected, including 2 mL of 30% acrylamide, 1.25 mL of 1.5M Tris hydrochloric acid, 50 μL of 10% SDS, 28 μL of 10% AP, 2.5 μL of TEMED, 1.675 μL of ddH₂O, and an overall volume of 5 mL. Leakage was tested with ddH₂O and then the ddH₂O was poured off, the remaining water was adsorbed with filter paper. Add the lower layer to the appropriate height and flatten with 100% alcohol, leave it at room temperature for 40 min, then pour off the excess alcohol and dry it with filter paper.

b) 4% concentrated gel was prepared, including 0.25 g of 30% acrylamide, 0.625 mL of 0.5M Tris hydrochloride, 25 μL of 10% SDS, 13 μL of 10% AP, TEMED 1.3 μL, and ddH₂O 1.525 μL. The total volume was 2.5 mL. The concentrated glue was poured into a glass plate and a comb with 10 or 15 holes was inserted as needed, and dried for t next experiment.

c) Pour the electrophoresis solution into the electrophoresis tank, pull out the comb, add samples into each hole, the amount of protein per hole was about 40 g; add the electrophoresis solution to the top of the electrophoresis tank, connect the electrode, perform 60 V electrophoresis at constant pressure for 45 min, adjust the voltage to 90 V after the sample presses the separation gel, continue electrophoresis until the bromophenol blue runs out of the lower layer of gel, and then disconnect the power supply. Carefully remove the separation gel and place it in the transfer solution to equilibrate for 15 minutes.

d) Cut the nitrocellulose membrane (PVDF) according to the size of the protein and transfer using the wet transfer method. Place sponge, filter paper, PVDF membrane, separation gel, filter paper, and sponge in sequence to ensure that there are no bubbles between the gel and the membrane. Fix the clamp and place it in the wet transfer tank. The transfer solution was poured and ice cubes were placed in the wet transfer tank, and transferred at a constant flow rate of 200 mA for 90 minutes.

e) After the transfer printing was completed, the PVDF membrane was taken out and placed in 5% skim milk powder or 5% BSA blocking solution, and placed on a shaker at room temperature for blockage for 1-2 hours.

f) The primary antibody was diluted according to the antibody instructions, and incubated with PVDF membrane for 14 hours. The primary antibody was recovered and washed 5-6 times with TBST for 5 minutes each time. After washing, the secondary antibody was incubated using HRP labeled antibodies of the corresponding species. The secondary antibody was diluted and dissolved proportionally in 5% skim milk powder, and incubated at room temperature for 1-1.5 hours. Thereafter, the antibody was washed again 5-6 times with TBST, each time for 5 minutes.

g) According to the instructions of the ECL luminescent reagent kit, the solution A and solution B were mixed in a 1:1 volume ratio. The luminescence detection was performed through an imaging system, and the results were quantitatively analyzed using Image J software.

### 8. Statistical Analysis

Statistical data were analyzed using SPSS 19.0 software and expressed as mean±S. E. M. Differences between groups were analyzed using Two-way ANOVA or One-way ANOVA combined with Turkey multiple comparisons. p<0.05 indicates statistical significance, and p<0.01 is considered highly significant.

It will be obvious to those skilled in the art that changes and modifications may be made, and therefore, the aim in the appended claims is to cover all such changes and modifications.

What is claimed is:

1. A method for preventing or treating Parkinson's disease comprising administering a patient in need thereof a pharmaceutical composition comprising a polypeptide, the peptide having an amino acid sequence I or II:
I: FPGSDRF (SEQ ID NO: 1)-Z;
II: X-FPGSDRF (SEQ ID NO: 1)-Z;
wherein:
S represents phosphorylated serine;
X and Z independently represents an amino acid or an amino acid sequence;
X is selected from F, (R)₉ (SEQ ID NO: 2), (R)₉-F (SEQ ID NO: 3), 6-aminohexanoic acid, 6-aminohexanoic acid-F, 6-aminohexanoic acid-(R)₉ (SEQ ID NO: 2), 6-aminohexanoic acid-(R)₉-F (SEQ ID NO: 3); and
Z is selected from (G)ₙ-RGD or A-(G)ₙ-RGD (SEQ ID NO: 4), where n is an integer greater than or equal to 0, in the range of 0-10.

2. The method of claim 1, wherein a symptom of the Parkinson's disease is excessive activation or proliferation of astrocytes or microglia in brain tissue.

3. The method of claim 1, wherein a symptom of the Parkinson's disease is degeneration or loss of dopamine neurons in brain tissue.

4. The method of claim 1, wherein a symptom of the Parkinson's disease is weakened vitality of hippocampal neurons or glial cells due to cytotoxic effects.

5. The method of claim 1, wherein a symptom of the Parkinson's disease is increased expression of inflammasomes and increased apoptosis in hippocampal neurons or glial cells.

6. The method of claim 1, wherein a symptom of the Parkinson's disease is increased membrane potential and elevated intracellular levels of reactive oxygen due to damage of cellular mitochondria in hippocampal neuronal cells or neuroglial cells.

7. The method of claim 1, wherein the polypeptide comprises an acetylated N-terminal and an amidated C-terminal.

8. The method of claim 1, wherein the polypeptide has an amino acid sequence of FPGSDRF-RGD, and serine (S) of the amino acid sequence of FPGSDRF-RGD is phosphorylated.

9. The method of claim 1, wherein the pharmaceutical composition further comprises a pharmaceutically acceptable excipient.
